(19) United States
(12) Patent Application Publication
(10) Pub. No.: US 2023/0181305 A1
(43) Pub. Date: Jun. 15, 2023

(54) GALLBLADDER IMPLANT

(71) Applicants: Boston Scientific Scimed, Inc., Maple Grove, MN (US); Regents of the University of Minnesota, Minneapolis, MN (US)

(72) Inventors: Ajay Gupta, Minneapolis, MN (US); Christian Angelopoulos, Minneapolis, MN (US); Sergei Grishankov, Minneapolis, MN (US); Mohamed Essa, Minneapolis, MN (US)

(73) Assignees: Boston Scientific Scimed, Inc., Maple Grove, MN (US); Regents of the University of Minnesota, Minneapolis, MN (US)

(21) Appl. No.: 18/082,254

(22) Filed: Dec. 15, 2022

Related U.S. Application Data
(60) Provisional application No. 63/289,717, filed on Dec. 15, 2021.

(51) Int. Cl. A61F 2/04

(52) U.S. Cl. CPC A61F 2/04; A61F 2002/041; A61F 2250/0067; A61F 2230/0008

(57) ABSTRACT

A medical implant is adapted to be implantable within a gallbladder in order to limit movement of any gallbladder stones present within the gallbladder. The medical implant includes a resilient body that is adapted to fit within the interior volume of the gallbladder and a plurality of pores that are formed within the resilient body. The pores are adapted to allow bile to flow through the pores.

### CROSS-REFERENCE TO RELATED APPLICATION

This application claims the benefit of priority under 35 U.S.C. § 119 of U.S. Provisional Application No. 63/289,717, filed Dec. 15, 2021, the entire disclosure of which is hereby incorporated by reference.

### TECHNICAL FIELD

The present disclosure pertains to medical devices, and methods for manufacturing and using medical devices. More particularly, the disclosure is directed to an implant for implantation within a patient's gallbladder.

### BACKGROUND

A wide variety of medical devices have been developed for medical use, for example, for use in accessing body cavities and interacting with fluids and structures in body cavities. Some of these devices may include guidewires, catheters, pumps, motors, controllers, filters, grinders, needles, valves, and delivery devices and/or systems used for delivering such devices. These devices are manufactured by any one of a variety of different manufacturing methods and may be used according to any one of a variety of methods. Of the known medical devices and methods, each has certain advantages and disadvantages.

### SUMMARY

This disclosure provides design, material, manufacturing method, and use alternatives for medical devices. As an example, a medical implant is adapted to be implantable within a gallbladder in order to limit movement of any gallbladder stones present within the gallbladder, the gallbladder defining an interior volume. The medical implant includes a resilient body adapted to fit within the interior volume of the gallbladder and a plurality of pores formed within the resilient body. The pores are adapted to allow bile to flow through the pores.

Alternatively or additionally, the resilient body may be adapted to allow natural movement of the gallbladder.

Alternatively or additionally, the resilient body may be adapted to be compressed for delivery into the gallbladder, and to expand once present within the gallbladder.

Alternatively or additionally, the resilient body may be adapted to at least substantially fill an interior volume of the gallbladder.

Alternatively or additionally, the resilient body may be adapted such that two or more medical implants in combination at least substantially fill an interior volume of the gallbladder.

Alternatively or additionally, the plurality of pores may include an average pore size of about 2 millimeters.

Alternatively or additionally, at least some of the plurality of pores may range from a minimum pore size of about 0.5 millimeters to a maximum pore size of about 5 millimeters.

Alternatively or additionally, the resilient body may include at least about 10 percent void space.

Alternatively or additionally, the resilient body may have a substantially uniform density throughout the body.

Alternatively or additionally, the resilient body may have a hollow interior.

Alternatively or additionally, the medical implant may further include a material disposed within the resilient body or over an exterior surface of the resilient body.

Alternatively or additionally, the material may include a pharmaceutical composition.

Alternatively or additionally, the material may include a radiopaque material.

Alternatively or additionally, the material may include a protective coating that protects an interior of the gallbladder.

As another example, a gallbladder implant is disclosed. The gallbladder implant includes a resilient body that is adapted to fit within an interior volume of a gallbladder, the resilient body adapted to allow bile to flow through the resilient body while restricting movement of any gallbladder stones present within the gallbladder.

Alternatively or additionally, the resilient body may include an open cell foam.

Alternatively or additionally, the resilient body may include a machined metallic or polymeric body.

Alternatively or additionally, the resilient body may include an ovoid shape.

Alternatively or additionally, the resilient body has a distal region and a proximal region, and the proximal region of the resilient body may have a cross-sectional profile larger than a cross-sectional profile of the distal region.

As another example, a gallbladder implant is disclosed. The gallbladder implant includes a resilient body that is adapted to fit within an interior volume of a gallbladder and a plurality of void spaces that are formed within the resilient body, the plurality of void spaces adapted to allow bile to flow through the void spaces, the plurality of void spaces restricting movement of any gallbladder stones present within the gallbladder.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a schematic illustration of the anatomy around the gallbladder;

FIG. 2 is a schematic illustration of a gallbladder;

FIG. 3 is a schematic illustration of a gallbladder with an illustrative gallbladder implant disposed within the gallbladder;

FIG. 4 is a close-up side view of a portion of an illustrative gallbladder implant;

FIG. 5 is a close-up side view of a portion of an illustrative gallbladder implant;

FIG. 6 is a side view of an illustrative gallbladder implant;

FIG. 7 is a close-up side view of a portion of an illustrative gallbladder implant;

FIG. 8 is a side view of an illustrative gallbladder implant;

FIG. 8A is an enlargement of a portion of the illustrative gallbladder implant of FIG. 8;

FIG. 9 is a side view of an illustrative gallbladder implant;

FIG. 10 is a cross-sectional view taken along the line 10-10 in FIG. 9; and

FIGS. 11 through 13 show an illustrative method of deploying an illustrative gallbladder implant.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

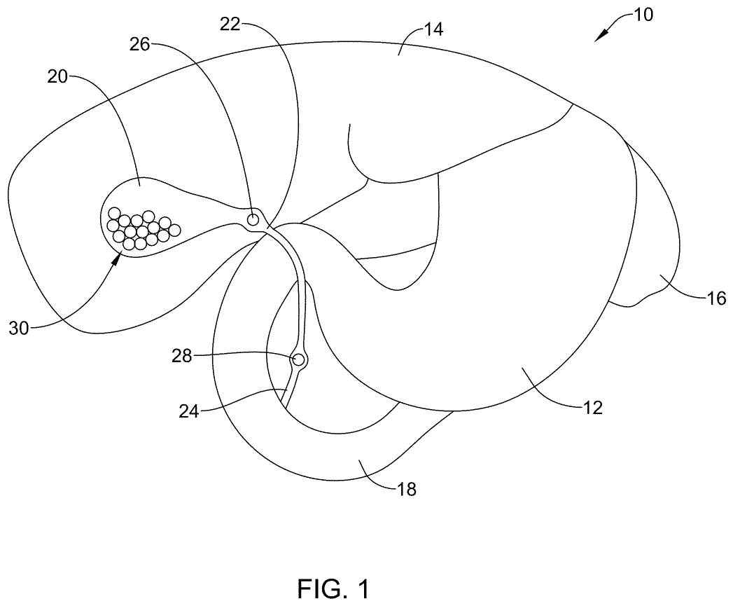

FIG. 1 is a schematic illustration of an anatomy 10 that shows a portion of a digestive tract of a patient. The anatomy 10 includes a stomach 12 and a liver 14. A spleen 16 is visible behind the stomach 12. The stomach 12 continues into a duodenum 18. A gallbladder 20 can be seen underneath the liver 14. The gallbladder 20 serves to store bile, which is produced in the liver 14 and which is released by the gallbladder 20 in order to help digest fatty foods. A cystic duct 22 extends from the gallbladder 20, as does a common bile duct 24. As illustrated, the cystic duct 22 includes a gallstone 26 that is currently blocking the cystic duct 22. The common bile duct 24 includes a gallstone 28 that is currently blocking the common bile duct 24. In this illustration, the gallbladder 20 is inflamed, and thus includes a number of gallstones 30. In some cases, gallstones that have reached one of the ducts, such as the gallstone 26 blocking the cystic duct 22 or the gallstone 28 blocking the common bile duct 24, are generally removed endoscopically.

The gallstones 30 within the gallbladder 20 can move. When the gallstones 30 move, they can irritate the bladder wall, causing inflammation and pain (cholecystitis). If they migrate into the ducts 22, 24, they can cause acute pain. In some cases, cholecystitis is treated by surgically removing the patient's gallbladder 20. In some cases, a patient is not a good candidate for surgery, for a variety of different reasons. For surgical patient's, gallbladder removal has a 0.5 to 2 percent complication rate. Gallbladder removal can cause sequalae-like post-cholecystectomy syndrome.

In general, gallstones 30 form when insoluble bile contents harden. There are two types of gallstones, cholesterol stones and pigmented stones. Cholesterol stones are formed when cholesterol concentrations are too much for available bile to dissolve. The cholesterol can aggregate or settle and combine with calcium carbonate, phosphates and bilirubin to form stones. In general, cholesterol stones are usually varying shades of yellow on their exterior and are black or gray on the inside.

Pigmented brown stones are a combination of calcium salts of unconjugated bilirubin and varying amounts of cholesterol and protein. Pigmented brown stones are generally formed as a result of a bacterial or parasitic infection of the bile ducts. Pigmented black stones are formed of calcium bilirubinate and are typically formed in cases of cirrhosis, hemolytic syndromes such as sickle cell anemia, and conditions of stasis.

It is estimated that 15 percent of the population will have gallstone disease at some point in their life. It can be difficult to treat the underlying causes for the formation of gallstones. If the disease progresses, the gallstones can be become symptomatic. If the gallstones migrate to the ducts, they can cause colic, infection, pancreatitis, among other problems, and can require emergency surgery. Some patients are unwilling to have surgery or have inhibitions against having an organ removed. Some cholecystectomies are considered high risk due to abnormal anatomies, lack of diagnostic equipment or even insufficient surgical training.

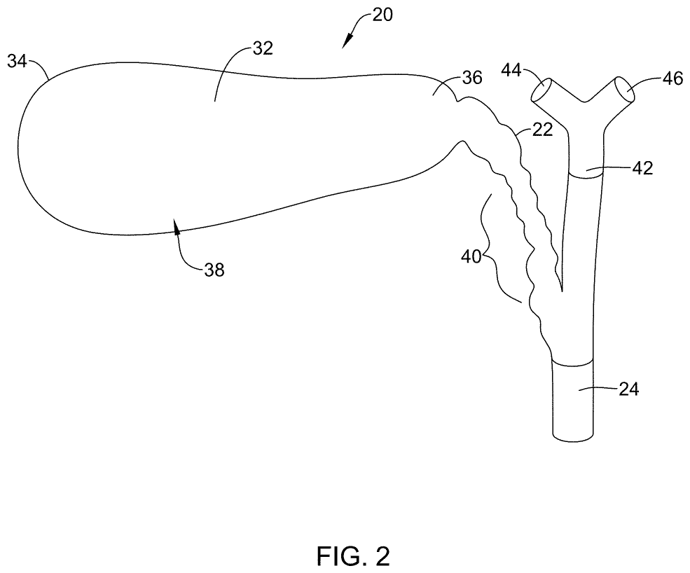

FIG. 2 shows additional details pertaining to the gallbladder 20. The gallbladder 20 can be considered as including a body 32, a fundus 34 (proximal end of the gallbladder 20) and a neck 36 (distal end of the gallbladder 20). As can be seen, the gallbladder 20 defines an interior volume 38 that consumes a majority of the gallbladder 20. Essentially, the gallbladder 20 is an empty organ that receives, stores and periodically releases bile. It will be appreciated that if one or more gallstones form within the gallbladder 20, they would be able to move around within the gallbladder 20. A spiral valve 40 extends between the cystic duct 22 and the common bile duct 24. A common hepatic duct 42 includes a right hepatic duct 44 and a left hepatic duct 46.

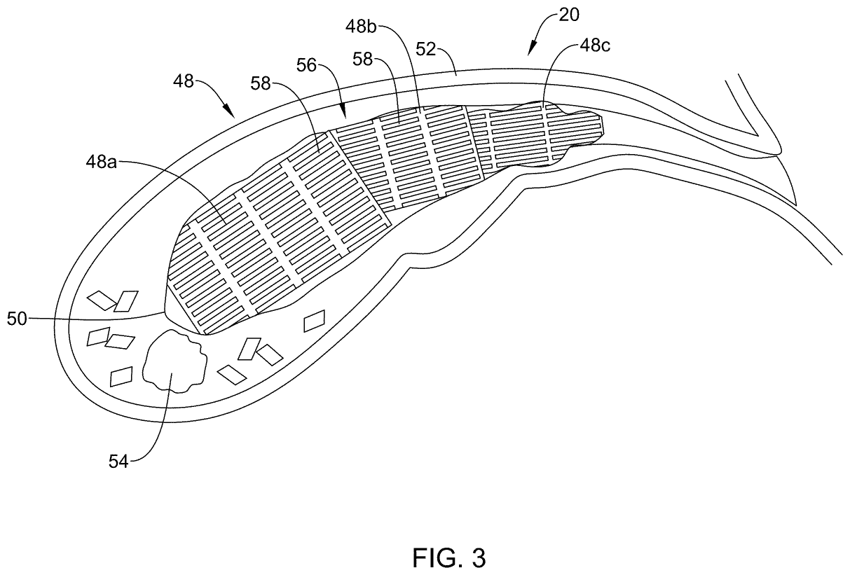

FIG. 3 shows a gallbladder implant 48 disposed within the gallbladder 20. In some cases, at least a portion of the interior volume 38 may be considered as forming a gallbladder lumen 50. A gallbladder wall 52 forms an exterior of the gallbladder 20. Mucin gel, sometimes referred to as “biliary sludge”, fills a part of the volume between the gallbladder wall 52 and the gallbladder lumen 50, as does (as shown) a gallstone 54. By filling the gallbladder lumen 50, the gallstone 54 is largely restricted from movement. Gallstones that do not move can't block a duct, or otherwise cause problems.

The gallbladder implant 48 may be beneficial for several categories of patients. For example, the gallbladder implant 48 may be beneficial for patients who cannot undergo surgery. For these patients, the gallbladder implant 48 provides a permanent solution, avoiding a need for continuous percutaneous drainage and a risky surgical procedure. For patients who do not wish to have their gallbladder 20 removed, but are at elevated risk for a gallbladder attack from a loose gallstone, these patients would be protected from a gallbladder attack without requiring permanent dietary changes as a result of otherwise having their gallbladder 20 removed. In some instances, use of the gallbladder implant 48 may eventually become the standard of care, in place of gallbladder removal.

Implantation of the gallbladder implant 48 is a quick procedure that does not require general anesthesia. Implantation of the gallbladder implant 48 avoids post-cholecystomy syndrome and avoids surgery. Use of the gallbladder implant 48 maintains the function of the gallbladder 20, thereby requiring no dietary changes. In some cases, the gallbladder implant 48 may subsequently be removed, possibly taking trapped gallstones with it.

In some cases, the gallbladder implant 48 may be considered as including a first portion 48a, a second portion 48b and a third portion 48c. In some cases, the first portion 48a, the second portion 48b and the third portion 48c may be parts of a unitary gallbladder implant 48. In some cases, one or more of the first portion 48a, the second portion 48b and the third portion 48c may be considered as separate gallbladder implants 48. In some cases, one gallbladder implant 48 is adapted to fill the gallbladder lumen 50 by itself. In some cases, a physician may implant two or three, or more, distinct gallbladder implants 48 such as the first portion 48a, the second portion 48b and/or the third portion 48c, some of which may be of varying sizes, in order to best fill the space within the gallbladder lumen 50.

The gallbladder implant 48 may be considered as having a resilient body 56 with a plurality of pores 58. In some cases, the resilient body 56 may be considered as being sufficiently compressible to be compressed into a substantially smaller shape for implantation. The resilient body 56 may be considered as being sufficiently resilient to allow for natural movement of the gallbladder 20, such as the contractions that the gallbladder 20 undergoes when releasing bile, without the gallbladder implant 48 negatively affecting the performance of the gallbladder 20.

The plurality of pores 58 may be considered as being adapted to permit fluids within the gallbladder 20, such as bile, to flow through the plurality of pores 58. This means that the presence of the gallbladder implant 48 does not impact, or does not substantially impact, the ability of the gallbladder 20 to store bile and to release bile in order to help digest fatty food. In some cases, the pores 58 are adapted to not allow gallstones to pass through the pores 58.

For example, the plurality of pores 58 may have an average pore size of about 2 millimeters (mm). At least some of the plurality of pores 58 may range from a minimum pore size of about 0.5 mm to a maximum pore size of about 5 mm. It will be appreciated that these pore sizes are applicable to at least a portion of the resilient 56 that is adapted to filter and immobilize gallstones. In some cases, the resilient body 56 may be considered as including at least about 10 percent void space, or at least about 20 percent void space, or at least about 30 percent void space, or at least about 40 percent void space, or at least about 50 percent void space, or more.

In some cases, the plurality of pores 58 may be uniformly distributed throughout the resilient body 56. In some instances, there may be relatively more pores 58 biased towards a portion of the resilient body 56 that is adapted to fit within the neck 36 of the gallbladder 20, near where the cystic duct 22 meets the gallbladder 20. In some instances, the portion of the resilient body 56 including a relatively greater number of pores 58 can function as a filter while a remaining portion of the resilient body 56 may have large pores or even a shell-shaped strut structure that is adapted to secure the resilient body 56 in position relative to the body 32 and the fundus 34 of the gallbladder 20.

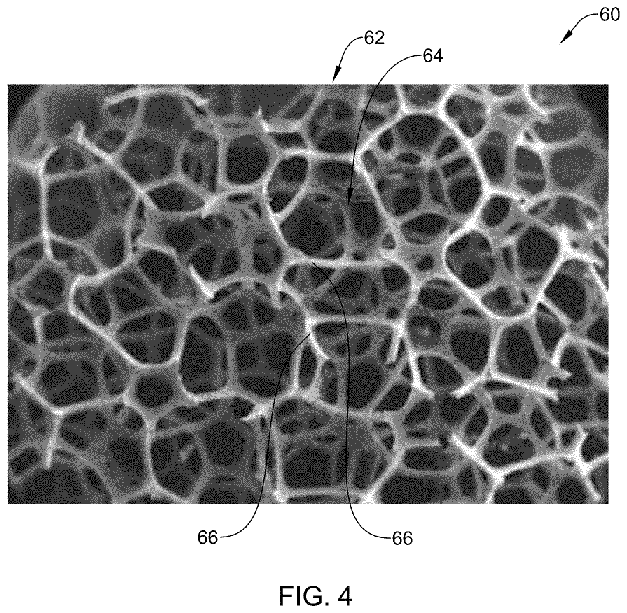

In some instances, the gallbladder implant 48 may have a reticulated structure that is formed as a machined body 56. In some cases, the gallbladder implant 48 may have a reticulated structure that is formed of an open cell foam, or other structure. In some cases, the resilient body 56 may have the same or substantially the same structure throughout, with a uniform density. In some cases, the resilient body 56 may form a shell, with a hollow interior of the shell. FIGS. 4 through 10 provide illustrative but non-limiting examples of various gallbladder implants.

FIG. 4 is a schematic close-up view of a portion of an illustrative gallbladder implant 60. The gallbladder implant 60 may, for example, be used in place of the gallbladder implant 48 shown in FIG. 3. The gallbladder implant 60 has a resilient body 62 with a plurality of pores 64 formed within the resilient body 62. As shown, the resilient body 62 may be considered as being formed of an open cell foam, such as but not limited to a polyurethane open cell foam, in which the resilient body 62 includes a number of struts 66 that define the pores 64 between adjacent struts 66. Open cell foams are generally formed by incorporating a blowing agent such as sodium bicarbonate into the material that forms the foam. This results in a complex structure in which adjoining pores 64 can be open to each other. The resilient body 62 is an example of a resilient body that likely has a uniform or substantially uniform structure and density throughout.

It will be appreciated that an open cell foam such as that shown forming the gallbladder implant 60 may be considered as having a large void fraction, meaning that the foam is mostly open space, rather than polyurethane. Having a large void fraction means that the gallbladder implant 60 can easily accommodate a significant volume of bile there within. Having a large void fraction also means that the gallbladder implant 60 can easily accommodate natural movement of the gallbladder 20. Having a large void fraction also means that the gallbladder implant 60 can easily be compressed for delivery.

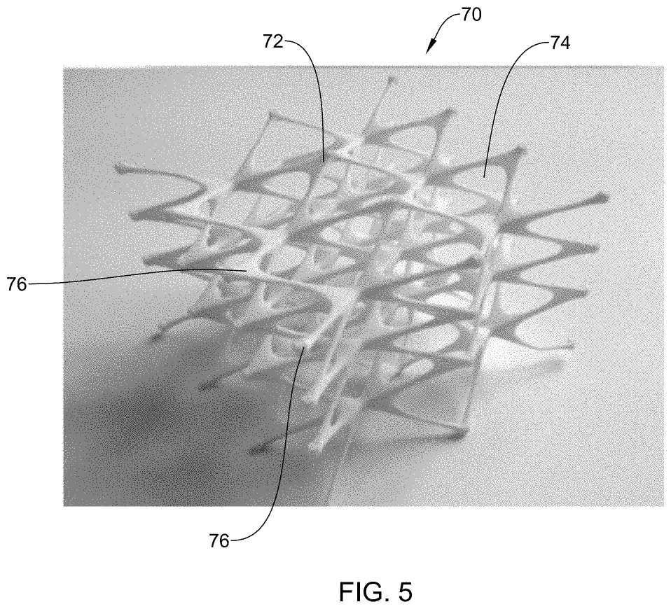

FIG. 5 is a schematic close-up view of a portion of an illustrative gallbladder implant 70. The gallbladder implant 70 may, for example, be used in place of the gallbladder implant 48 shown in FIG. 3. The gallbladder implant 70 has a resilient body 72 with a plurality of pores 74 formed within the resilient body 72. As shown, the resilient body 72 may be considered as being formed from an octagonal repeating unit in which at least some crossmembers have been removed. The resilient body 72 may be formed from a metallic or polymeric repeating unit, for example, and includes a number of struts 76. The resilient body 72 is an example of a resilient body that likely has a uniform or substantially uniform structure and density throughout.

It will be appreciated that the resilient body 72 may be considered as having a large void fraction, meaning that the resilient body 72 is mostly open space, rather than material. Having a large void fraction means that the gallbladder implant 70 can easily accommodate a significant volume of bile therewithin. Having a large void fraction also means that the gallbladder implant 70 easily accommodate natural movement of the gallbladder 20. Having a large void fraction also means that the gallbladder implant 70 can easily be compressed for delivery.

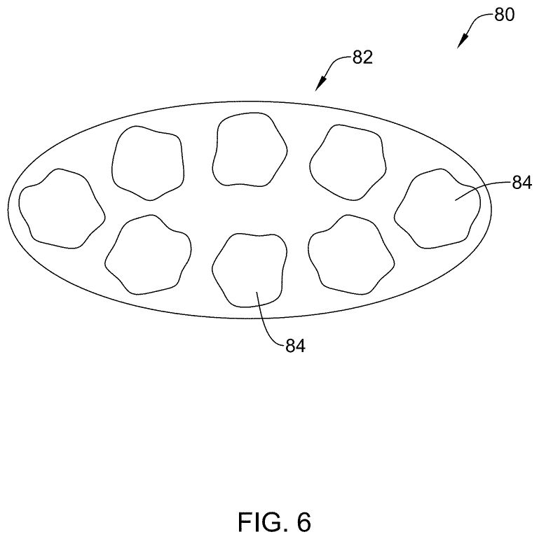

FIG. 6 is a schematic view of an illustrative gallbladder implant 80. The gallbladder implant 80 may, for example, be used in place of the gallbladder implant 48 shown in FIG. 3. The gallbladder implant 80 has a resilient body 82 with a plurality of voids 84 formed within the resilient body 82. As shown, the resilient body 82 may be considered as being a sponge, in which the voids 84 allow bile to be stored within the resilient body 82 and to pass through the resilient body 82. In some cases, the plurality of voids 84 may be smaller, and more numerous, than what is illustrated. The resilient body 82 is an example of a resilient body that likely has a uniform or substantially uniform structure and density throughout.

It will be appreciated that the resilient body 82 may be considered as having a large void fraction, meaning that the resilient body 82 is mostly open space, rather than material. Having a large void fraction means that the gallbladder implant 80 can easily accommodate a significant volume of bile therewithin. Having a large void fraction also means that the gallbladder implant 80 easily accommodate natural movement of the gallbladder 20. Having a large void fraction also means that the gallbladder implant 80 can easily be compressed for delivery.

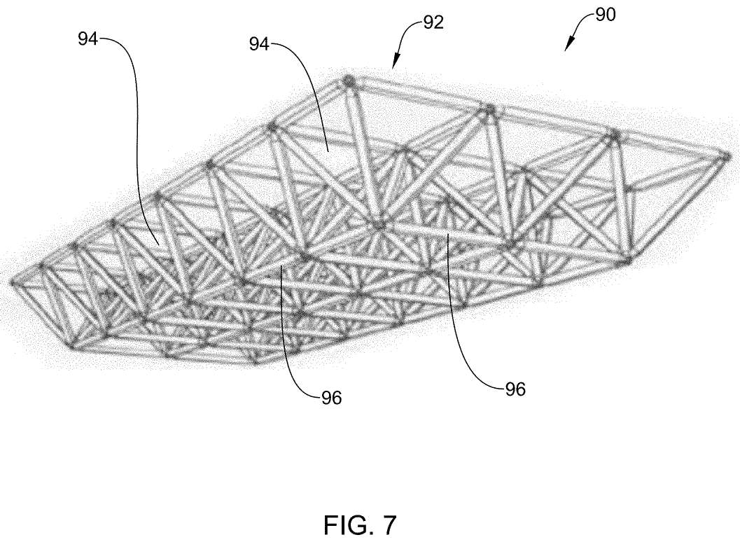

FIG. 7 is a schematic close-up view of a portion of an illustrative gallbladder implant 90. The gallbladder implant 90 may, for example, be used in place of the gallbladder implant 48 shown in FIG. 3. The gallbladder implant 90 has a resilient body 92 with a plurality of pores 94 formed within the resilient body 92. As shown, the resilient body 92 may be considered as being machined or otherwise formed from a plurality of struts 96 that together define the pores 94.

It will be appreciated that the resilient body 92 may be considered as having a large void fraction, meaning that the resilient body 92 is mostly open space, rather than material. Having a large void fraction means that the gallbladder implant 90 can easily accommodate a significant volume of bile therewithin. Having a large void fraction also means that the gallbladder implant 90 easily accommodate natural movement of the gallbladder 20. Having a large void fraction also means that the gallbladder implant 90 can easily be compressed for delivery, with adjacent struts 96 pivoting to allow for compression, for example.

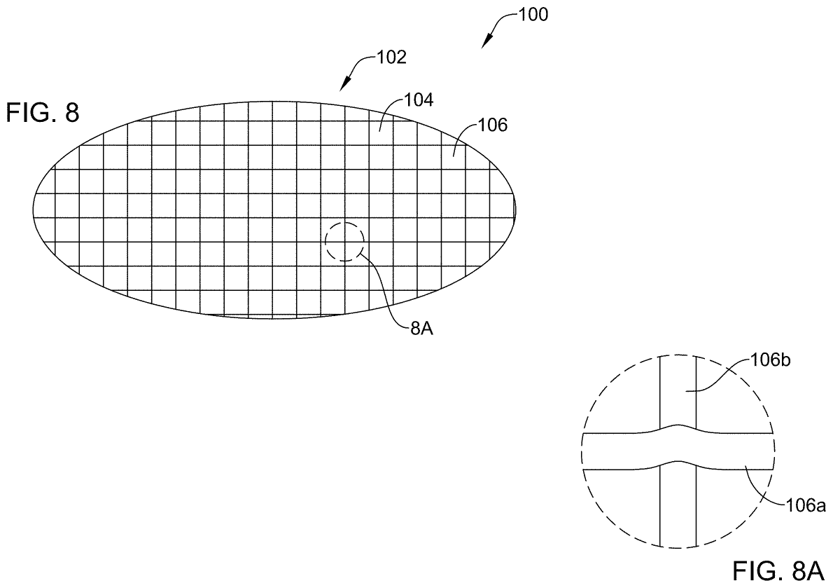

FIG. 8 is a schematic view of an illustrative gallbladder implant 100. The gallbladder implant 100 may, for example, be used in place of the gallbladder implant 48 shown in FIG. 3. The gallbladder implant 100 has a resilient body 102 with a plurality of pores 104 formed within the resilient body 102. As shown, the resilient body 102 may be considered as being woven or braided from one or more wires 106, as can be seen in FIG. 8A, which is an enlargement showing a first wire 106a passing over a second wire 106b. While the first wire 106a is shown as being orthogonal or substantially orthogonal to the second wire 106b, this is required in all cases, and the first wire 106a and the second wire 106b may pass at an acute angle, for example. The resilient body 102 is an example of a resilient body that likely forms a shell around an empty interior.

It will be appreciated that the resilient body 102 may be considered as having a large void fraction, meaning that the resilient body 102 is mostly open space, rather than material. Having a large void fraction means that the gallbladder implant 100 can easily accommodate a significant volume of bile therewithin. Having a large void fraction also means that the gallbladder implant 100 easily accommodate natural movement of the gallbladder 20. Having a large void fraction also means that the gallbladder implant 100 can easily be compressed for delivery.

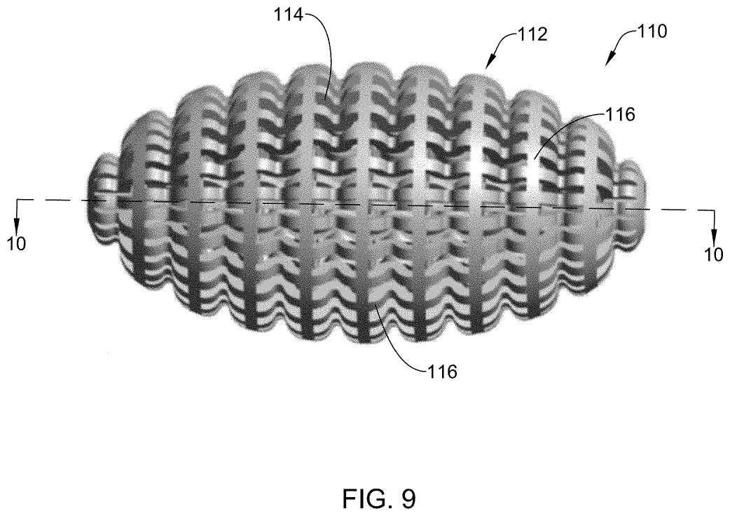

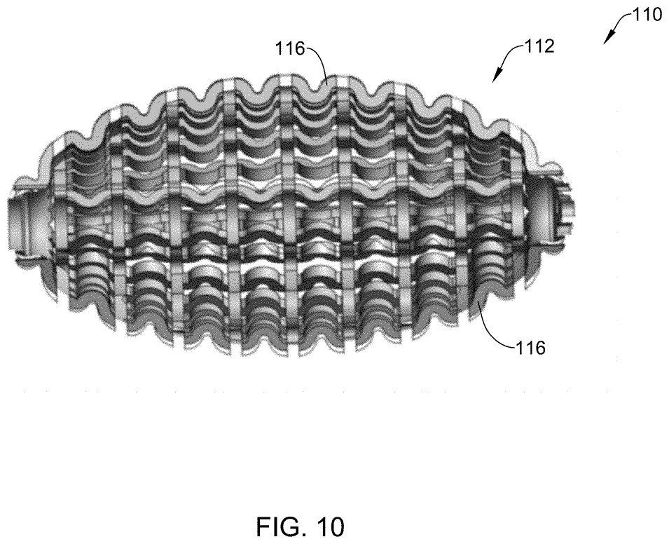

FIG. 9 is a schematic view of an illustrative gallbladder implant 110 and FIG. 10 is a cross-sectional view taken along line 10-10 of FIG. 9. The gallbladder implant 110 may, for example, be used in place of the gallbladder implant 48 shown in FIG. 3. The gallbladder implant 110 has a resilient body 112 with a plurality of pores 114 formed within the resilient body 112. As shown, the resilient body 102 may be considered as being machined from a metallic or polymeric body to define a number of struts 116 that together define the pores 114. The resilient body 102 may be laser cut, for example, and may be considered as being an example of a resilient body that likely forms a shell around an empty interior. In some cases, the gallbladder implant 110 may be considered as being an ellipsoid, which is generally a closed surface (discounting the pores 114) of which all plane cross-sections are either ellipses or circles.

It will be appreciated that the resilient body 112 may be considered as having a large void fraction, meaning that the resilient body 112 is mostly open space, rather than material. Having a large void fraction means that the gallbladder implant 110 can easily accommodate a significant volume of bile therewithin. Having a large void fraction also means that the gallbladder implant 110 easily accommodate natural movement of the gallbladder 20. Having a large void fraction also means that the gallbladder implant 110 can easily be compressed for delivery, with adjacent struts 116 pivoting to allow for compression, for example.

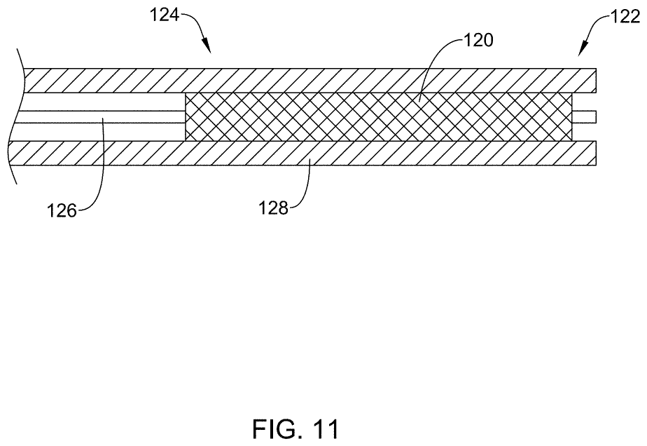

FIGS. 11 through 13 provide an illustrative but non-limiting example of deploying an illustrative gallbladder implant 120 that has a distal region 122 and a proximal region 124. It will be appreciated that any of the gallbladder implant 48, the gallbladder implant 60, the gallbladder implant 70, the gallbladder implant 80, the gallbladder implant 90, the gallbladder implant 100 or the gallbladder implant 110 may be delivered in a similar manner.

To deliver the gallbladder implant 120 (or any of the other gallbladder implants), ultrasound may be used to confirm that there are gallstones in the gallbladder 20, but no gallstones in the ducts. Ultrasound may also be used to confirm that macroscopically the gallbladder 20 appears normal, to discount the possibility of malignancy.

The cystic duct may be examined fluoroscopically to confirm duct patency. Access may be gained percutaneously or peritoneally to the gallbladder 20 by puncturing the gallbladder 20 with a needle under ultrasound guidance. This may include use of a 18G Chiba needle, for example. Any bile present within the gallbladder 20 may be drained to reduce distention and bile spillage into the peritoneum. A guidewire may be introduced, and the tract may be dilated using an introducer sheath. An 8F tube may be used, for example, but this is merely an example. Optionally, any small stones near the cystic duct entrance may be sucked out. Next, the gallbladder implant 120 may be delivered. In some cases, a gallbladder implant may be removed and replaced with a new gallbladder implant, such as when the first gallbladder implant contains a number of trapped gallstones, for example.

In FIG. 11, the gallbladder implant 120 is shown compressed onto an elongate member 126, which in some cases may be advanced over a guidewire (not shown). In this particular example, the gallbladder implant 120 may be considered as being a woven or braided gallbladder implant. An outer sheath 128 extends over the gallbladder implant 120 and holds the gallbladder implant 120 in a compressed configuration.

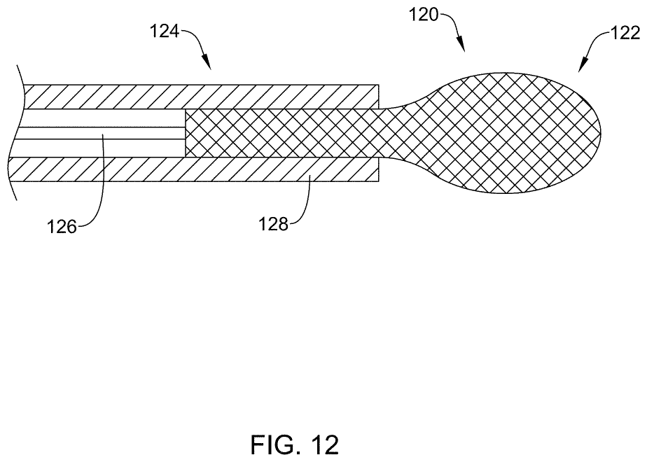

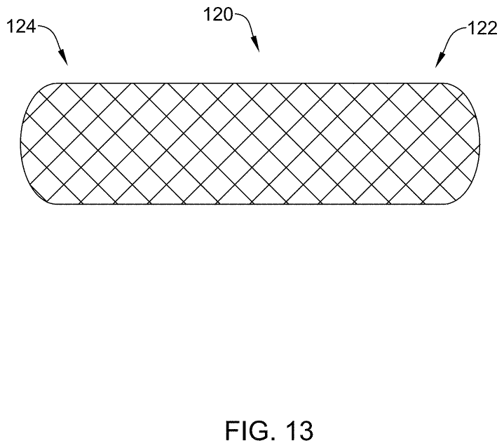

In FIG. 12, the outer sheath 128 has been withdrawn proximally, allowing the distal region 122 of the gallbladder implant 120 to begin to expand. Continuing to withdraw the outer sheath 128 will allow the gallbladder implant 120 to gain its fully expanded, deployed configuration, as shown in FIG. 13. In this example, the gallbladder implant 120 was self-expanding. In some cases, the gallbladder implant 120 may be pushed out of the outer sheath 128.

In some cases, the gallbladder implants 48, 60, 70, 80, 90, 100, 110, 120 may be delivered percutaneously. The gallbladder implants 48, 60, 70, 80, 90, 100, 110, 120 may be delivered laparoscopically or endoscopically using suitable devices. The gallbladder implants 48, 60, 70, 80, 90, 100, 110, 120 may be made in a variety of sizes. In some cases, the gallbladder implants 48, 60, 70, 80, 90, 100, 110, 120 may be trimmed to size before implantation.

The gallbladder implants 48, 60, 70, 80, 90, 100, 110, 120 may be formed of materials that have shape memory and sufficient resiliency to withstand repeated deformations, including that caused by delivery as well as subsequent movement (contraction) of the gallbladder 20. Suitable materials are non-toxic and able to withstand the bile environment, which is generally a high pH environment with enzymes and bacteria. Suitable materials are biocompatible. Examples of suitable materials include but are not limited to silicone, metal wires, urethanes, polyolefins, polyolefin elastomers, fluoropolymers, their copolymers, and combinations thereof.

The gallbladder implants 48, 60, 70, 80, 90, 100, 110, 120 may include agents to provide visibility under various imaging methodologies. This may include doping the material used to form the gallbladder implants 48, 60, 70, 80, 90, 100, 110, 120 with radiopaque fillers and markers, making the gallbladder implants 48, 60, 70, 80, 90, 100, 110, 120 visible under ultrasound, x-ray, MRI, fluorescence, or any other imaging modality.

In some cases, the gallbladder implants 48, 60, 70, 80, 90, 100, 110, 120 may be coated with or otherwise include materials that increase and improve the functionality of the gallbladder implants 48, 60, 70, 80, 90, 100, 110, 120. This may include materials that can prevent stone nucleation, provide anti-fouling capability, provide lubrication, and the like. This may also include pharmaceutical compositions that provide anti-inflammatory properties, and antibiotics. The gallbladder implants 48, 60, 70, 80, 90, 100, 110, 120 may include an outer layer of a perforated fluoropolymer film.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A medical implant adapted to be implantable within a gallbladder in order to limit movement of any gallbladder stones present within the gallbladder, the gallbladder defining an interior volume, the medical implant comprising:
a resilient body adapted to fit within the interior volume of the gallbladder; and
a plurality of pores formed within the resilient body;
wherein the pores are adapted to allow bile to flow through the pores.

2. The medical implant of claim 1, wherein the resilient body is adapted to allow natural movement of the gallbladder.

3. The medical implant of claim 1, wherein the resilient body is adapted to be compressed for delivery into the gallbladder, and to expand once present within the gallbladder.

4. The medical implant of claim 1, wherein the resilient body is adapted to at least substantially fill an interior volume of the gallbladder.

5. The medical implant of claim 1, wherein the resilient body is adapted such that two or more medical implants in combination at least substantially fill an interior volume of the gallbladder.

6. The medical implant of claim 1, wherein the plurality of pores comprise an average pore size of about 2 millimeters.

7. The medical implant of claim 1, wherein at least some of the plurality of pores range from a minimum pore size of about 0.5 millimeters to a maximum pore size of about 5 millimeters.

8. The medical implant of claim 1, wherein the resilient body comprises at least about 10 percent void space.

9. The medical implant of claim 1, wherein the resilient body has a substantially uniform density throughout the body.

10. The medical implant of claim 1, wherein the resilient body has a hollow interior.

11. The medical implant of claim 1, further comprising a material disposed within the resilient body or over an exterior surface of the resilient body.

12. The medical implant of claim 11, wherein the material comprises a pharmaceutical composition.

13. The medical implant of claim 11, wherein the material comprises a radiopaque material.

14. The medical implant of claim 11, wherein the material comprises a protective coating that protects an interior of the gallbladder.

15. A gallbladder implant, comprising:
a resilient body adapted to fit within an interior volume of a gallbladder, the resilient body adapted to allow bile to flow through the resilient body while restricting movement of any gallbladder stones present within the gallbladder.

16. The gallbladder implant of claim 15, wherein the resilient body comprises an open cell foam.

17. The gallbladder implant of claim 15, wherein the resilient body comprises a machined metallic or polymeric body.

18. The gallbladder implant of claim 15, wherein the resilient body comprises an ovoid shape.

19. The gallbladder implant of claim 15, wherein the resilient body has a distal region and a proximal region, and the proximal region of the resilient body has a cross-sectional profile larger than a cross-sectional profile of the distal region.

20. A gallbladder implant, comprising:
a resilient body adapted to fit within an interior volume of a gallbladder; and
a plurality of void spaces formed within the resilient body, the plurality of void spaces adapted to allow bile to flow through the void spaces, the plurality of void spaces restricting movement of any gallbladder stones present within the gallbladder.
